(19) United States
(12) Patent Application Publication
(10) Pub. No.: US 2006/0089400 A1
(43) Pub. Date: Apr. 27, 2006

(54) Novel spiro ketone and carboxylic acid derivatives as specific inhibitors for (po3h2) ser/(po3h2)thr-pro-specific peptidyl-prolylcis/trans-isomerases

(75) Inventors: Birte Hernandez Alvarez, Tubingen (DE); Gunter S Fischer, Halle (DE); Manfred Braun, Schifferstadt (DE); Anahita Hessamian-Alinejad, Bottrop (DE); Hans Fliri, Boxborough, MA (US)

Correspondence Address: LAHIVE & COCKFIELD, 28 STATE STREET, BOSTON, MA, 02109, US

(73) Assignee: The Vernalis Group of Companies, Winnersh (GB)

(21) Appl. No.: 10/514,279

(22) PCT Filed: May 2, 2003
(86) PCT No.: PCT/EP03/04623

§ 371 Date: Oct. 19, 2005

(30) Foreign Application Priority Data
May 3, 2002 (EP) 02009365.4

(51) Int. Cl. A61K 31/407; A61K 31/343; A61K 31/277; C07D 209/96; C07D 317/72

(52) U.S. Cl. 514/409; 514/453; 514/521; 514/567; 548/409; 549/336; 558/410; 562/459

(57) ABSTRACT

The invention provides new spiro and carboxylic acid derivatives, pharmaceutically compositions comprising them and their use for the preparation of pharmaceutical compositions.

The present invention relates to new low-molecular spiro, ketone and carboxylic acid compounds and their derivatives having an affinity for (PO₃H₂)Ser/(PO₃H₂)Thr-Pro-specific peptidyl-prolyl-cis/trans-isomerases of the parvuline family and which inhibit their enzymatic peptidyl-prolyl-cis/trans-isomerase activity, as well as pharmacologically acceptable salts and pharmaceutical compositions containing such compounds. Moreover, the invention relates to the use of the above-indicated compounds for the preparation of pharmaceutical compositions as therapeutics for the treatment of diseases characterised by disorders of the cell proliferation, such as e.g. in the case of cancer or infectious diseases.

Peptidyl-prolyl-cis/trans-isomerases are enzymes which can catalyse the interconversion of cis- and trans-isomers of peptidyl-prolyl bonds. There are three peptidyl-prolyl cis/trans-isomerase families: the cyclophilines, the FK506-binding proteins (FKBP) and the parvulines. The three known peptidyl-prolyl cis/trans-isomerase families differ from each other both with respect to the conservation of motifs within their amino acid sequences and with respect to the possibility of inhibiting them enzymatically by low-molecular natural substances [1-4]. The representatives of the cyplophilines and FKBP are reversibly inhibited in their enzymatic activity by the binding of the immunosuppressive substances Cyclosporin A (CsA) or FK506.

For several representatives of the parvuline-type of the peptidyl-prolyl-cis/trans-isomerases, the parvuline from Escherichia coli, the ESS1/PTF1 from Saccharomyces cerevisiae and the human Pin1, an irreversible inhibition by the natural substance juglone (5-hydroxy-1,4-naphtochinone) could be shown [5] which is specific among prolyl isomerases for parvulines. Juglone is a natural substance isolated from walnut with bacteriostatic and fungicidal as well as cytotoxic properties against eukaryotic cells [6,7].

Within the parvuline family, there are two groups of enzymes, which differ with respect to their substrate specificity. The first groups comprises all eukaryotic enzymes with a specificity for (PO₃H₂)Ser/(PO₃H₂)Thr-residues in front of the proline in the substrate. These include inter alia the human Pin1 (hPin1) and the ESS1/PTF1 from yeast [8-10]. Some prokaryotic as well as some of the eukaryotic which are known until now are not specific for phosphorylated substrates. They are summarised in the second group.

Reversible phosphorylation of Ser/Thr residues plays a central role in the regulation of basic cellular processes. The regulation of the eukaryotic cell cycle underlies, e.g. the principle of a sequence of activations of different signal transduction cascades which are very strictly adhered to with respect to time. This process is mainly directed by proline-specific Ser/Thr phosphatases and kinases. The reversible phosphorylation of proteins to Ser/Thr residues leads to structural changes of proteins and thus regulates their biologic activity, e.g. with respect to their stability, enzymatic activity or their binding affinity with respect to other proteins [11]. The peptidyl-prolyl bond also plays an important role when it comes to determining the three-dimensional protein structure which can have two different conformations, i.e. cis or trans.

The human Pin1 participates in the regulation of the eukaryotic cell cycle, particularly the mitosis. An overexpression of hPin1 in Hela cells leads to a blocking of the cell cycle in the transition from the G₂- to the M-phase, while its depletion results in a mitosis-arrested phenotype [8]. Different tests have shown that hPin1 binds a great number of mitotic phosphoproteins, as e.g. Cdc25, Weel, the large subunit of the RNA polymerase II and also hyperphosphorylated tau-protein [12-14]. During these tests, hints could be found with respect to a possible regulation of these proteins by the interaction with hPin1. It could be shown that Pin1 coming from the South American clawed frog Xenopus laevis as well as its homologue ESS1/PTF1 from yeast participates in the regulation of the transcription [15,16].

Human Pin1 is overexpressed in different breast cancer cell lines. It participates in the regulation of the transcription of the Cycline D1 gene as well as of the target genes of the β-catenine/APC signalling path [17,18]. Overexpression of Cycline D1 leads to a number of human cancerous diseases. A regulation of the interaction of APC with β-catenine could be shown for Pin1. Mutation in the APC gene leading to a change in the binding pattern of APC for β-catenine are, in turn, a common reason for cancerous diseases, as e.g. colon cancer.

It could be shown that due to the cis/trans isomerisation of (PO₃H₂)Ser/(PO₃H₂)Thr-Pro bonds in peptide substrates derived from the amino acid sequence of Cdc25, hPin1 influences their conformation and thus their accessibility for the conformation-dependent proline-specific phosphatase PP2A [19]. Thus, just as human Pin1 as (PO₃H₂)Ser/(PO₃H₂)Thr-Pro-specific peptidyl-prolyl-cis/trans-isomerases, exhibits an essential regulatory function in vivo.

The design of highly effective and specific pharmaceuticals for the regulation of mitotic processes is a main issue of the pharmaceutical industry. These pharmaceuticals could serve as effective chemotherapeutics in the treatment of diseases with errors in the regulation of cell proliferation, as e.g. cancer and infectious diseases.

Thus, the problem underlying the present invention is the provision of compounds which could be used in the therapy of the cited diseases.

Detection of peptidyl-prolyl cis/trans Isomerase Activities

As described in example 1, the peptidyl-prolyl-cis/trans-isomerase activity can be determined in the so-called protease coupled PPlase assay with the help of isomer-specific proteolysis and the suitable oligopeptide substrates and isomer-specific proteases [20]. Other processes for the detection of PPlase activity are e.g. observing isomer-specific differences with respect to spectroscopic properties, their mobilities or the foldback catalysis of other proteins as well as isomer-specific chemical compounds when recording core resonance spectra [21-24].

Inhibition of the PPlase Activity by Active Components

If adding active components to one of the test systems listed above leads to a measurable decrease in the reaction speed catalysed by the respective PPlase, these active components are classed PPlase inhibitors, as is exemplarily shown in example 1. Assessment of the PPlase specificity of the inhibitor is carried out by testing different PPlases in the assay. By varying the concentration of the inhibitor and assessing the results measured with conventional processes, the corresponding inhibition constant (K_{I}-value) can be determined [25]. The concentration of the inhibitor which inhibits the PPlase-induced catalysis by 50% is called IC₅₀-value.

### DETAILED DESCRIPTION

The present invention refers to a

1. A spiro derivative of the general formula (1) in all its enantiomeric forms: 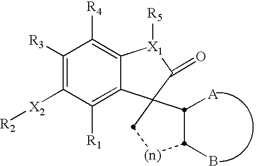
in which
X₁ represents —CH, —O— or nitrogen;
X₂ represents —CH₂, —O—, —NH—;
R₂ and R₅ represent a hydrogen atom or a linear or a branched C₁-C₈ alkyl group, which can be substituted by —OH, —OCH₃, —CH₂CH₂OCH₃, OCH₂CH₂—N(CH₃)₂ or —NH₂, C₁-C₈ alkylamino or C₁-C₈ dialkylamino groups;
or the moiety X₁-R₅ represents —O—;
R₁, R₃ and R₄ each independently represent a hydrogen atom, a linear or branched C₁-C₈ alkyl group, a linear or branched C₂-C₈ alkenyl group with one or several double bonds or a linear or branched C₂-C₈ alkinyl group with one or several triple bonds, a halogen atom selected from Cl, Br, I and F, a linear or branched C₁-C₈ acyl group or a C₁-C₈ amidoacyl group having the formula H₂NC(O)— or —HNC(O)—;
R₃ and R₄ and R₄ and R₅ can, additionally, together form a 5- or 6-membered aromatic or heteroaromatic ring, i.e. a ring substituted with nitrogen, oxygen or sulphur, which can additionally contain F, Cl, Br, I, CN, NO₂, —SH, O and —C(O)H;
(n) and the broken line represent a 4- (n=0), 5- (n=1), 6- (n=2) or 7- (n=3)-membered ring, wherein, except for the spiro carbon atom, each of the ring atoms . can individually and independently from each other represent a C—, N— or O— atom and these atoms can, except for oxygen, be linked by single as well as by double bonds, if C is the ring atom, it can be substituted by a linear or branched C₁-C₈ alkyl group, a linear or branched C₂-C₈ alkenyl group with one or several double bonds, a linear or branched C₂-C₈ alkinyl group with one or several triple bonds, a keto group, —OH, —OCH₃, —CH₂CH₂OCH₃, OCH₂CH₂—N(CH₃)₂ or by NH₂—, C₁-C₈ alkylamino or C₁-C₈ dialkylamino groups, if N is the ring atom, it can additionally have a hydrogen atom or it can be substituted by amino-, C₁-C₈ alkylamino or dialkylamino groups;
A and B represent either H or a 5- to 7-membered aromatic, heteroaromatic or saturated ring system; as well as pharmaceutically acceptable salts thereof.

2. A spiro derivative of the general formula (2) in all its enantiomeric forms: 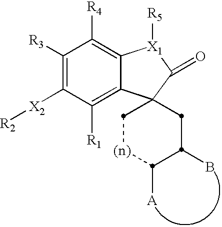
in which R₁, R₂, R₃, R₄, R₅, X₁, X₂, A and B are as defined under item 1;
(n) and the broken line represent a 5- (n=0), 6- (n=1), 7- (n=2), or 8- (n=3)-membered ring, wherein, except for the spiro carbon atom, each of the ring atoms . can individually and independently from each other represent a C-, N- or O-atom and these atoms can, except for oxygen, be linked by single as well as by double bonds, if C is the ring atom, it can be substituted by a linear or branched C₁-C₈ alkyl group or a linear or branched C₂-C₈ alkenyl group with one or several double bonds, a linear or branched C₂-C₈ alkinyl group with one or several triple bonds, a keto group, —OH, —OCH₃, —CH₂CH₂OCH₃, OCH₂CH₂—N(CH₃)₂ or by NH₂—, C₁-C₈ alkylamino or C₁-C₈ dialkylamino groups, if N is the ring atom, it can additionally have a hydrogen atom or it can be substituted by amino-, C₁-C₈ alkylamino or dialkylamino groups as well as pharmaceutically acceptable salts thereof.

3. A carboxylic acid derivative of the general formula (3) in all its enantiomeric forms: 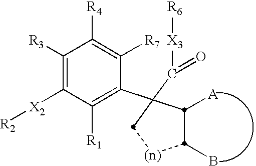
in which
X₃ represents O or NH;
X₂ represents —CH₂, —O— or —NH—;
R₁, R₂, R₃, R₄, (n), A and B are as defined under item 1;
R₆ and R₇ each independently represent a hydrogen atom, a linear or branched C₁-C₈ alkyl group, a linear or branched C₂-C₈ alkenyl group with one or several double bonds, or a linear or branched C₂-C₈ alkinyl group with one or several triple bonds, a halogen atom selected from Cl, Br, I and F, a linear or branched C₁-C₈ acyl group, or a C₁-C₈ amidoacyl group having the formula H₂NC(O)— or —HNC(O)—;
R₃ and R₄ and R₄ and R₇ can, additionally, together form a 5- or 6-membered aromatic or heteroaromatic ring, i.e. a ring substituted with nitrogen, oxygen or sulphur, which can additionally contain F, Cl, Br, I, CN, NO₂, —SH, O and —C(O)H as well as pharmaceutically acceptable salts thereof.

4. A carboxylic acid derivative of the general formula (4) and in all its enantiomeric forms: 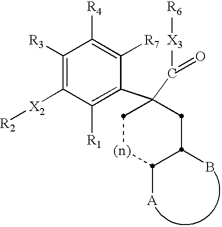
in which X₂, X₃, R₁, R₂, R₃, R₄, R₆, R₇, A and B are as defined under item 3;
(n) is as defined under item 2 as well as pharmaceutically acceptable salts thereof.

5. A Spiro derivative of the general formula (5) in all its enantiomeric forms: 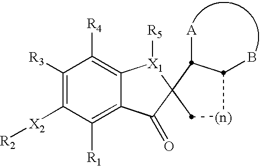
in which
X₁, X₂, R₁, R₂, R₃, R₄, R₅, (n), A and B are as defined under item 1.

6. A spiro derivative of the general formula (6) in all its enantiomeric forms: 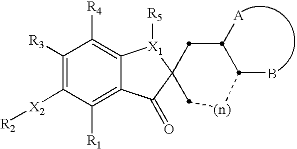
in which
X₁, X₂, R₁, R₂, R₃, R₄, R₅, A, B and (n) are as defined under item 2 as well as pharmaceutically acceptable salts.

7. A ketone derivative of the general formula (7) in all its enantiomeric forms: 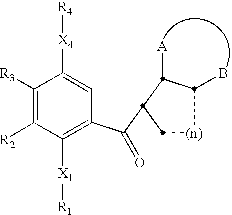
in which
X₁ and X₄ represent —CH₂, —O— or —NH—;
R₁ and R₄ represent a hydrogen atom or a linear or branched C₁-C₈ alkyl group, which can be substituted by —OH, —OCH₃, —CH₂CH₂OCH₃, —OCH₂CH₂—N(CH₃)₂ or by —NH₂—, C₁-C₈ alkylamino or C₁-C₈ dialkylamino groups;
R₂ represents independently a hydrogen atom, a linear or branched C₁-C₈ alkyl group, a linear or branched C₂-C₈ alkenyl group with one or several double bonds or a linear or branched C₂-C₈ alkinyl group with one or several triple bonds, a halogen atom selected from Cl, Br, I and F, a linear or branched C₁-C₈ acyl group, or a C₁-C₈ amidoacyl group having the formula H₂NC(O)— or —HNC(O)—;
R₂ and R₃ can, additionally, together form a 5- or 6-membered aromatic or heteroaromatic ring, i.e. a ring substituted with nitrogen, oxygen or sulphur, which can additionally contain F, Cl, Br, I, CN, NO₂, —SH, O and —C(O)H; and
R₃, (n), A and B are as defined under item 1 as well as pharmaceutically acceptable salts thereof.

8. A ketone derivative of the general formula (8) in all its enantiomeric forms: 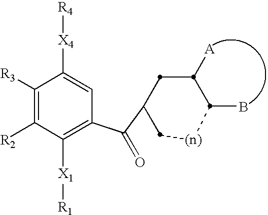
in which
X₁, X₄, R₁, R₂ and R₄ are as defined under item 7;
R₃, (n), A and B are as defined under item 2 as well as pharmaceutically acceptable salts thereof.

In a preferred embodiment, the compounds according to the invention have a molecular weight which is lower than about 1,000 g/Mol, preferably lower than 750 g/Mol and more preferably lower than about 500 g/Mol.

The present invention further provides pharmaceutical compositions comprising the compound according to the invention optionally coupled to pharmaceutical carrier molecules, as e.g. amino acids or oligopeptides, its pharmaceutically acceptable salts.

In the above-indicated formulae of the compounds according to the invention and in the following “alkyl” represents a linear or branched alkyl group, particularly C₁-C₈ alkyles as methyl, ethyl, propyl, iso-propyl, butyl, sec-butyl, iso-butyl, tert-butyl, n-pentyl, n-hexyl, etc. or an alkyl substituted by an aryl, heteroaryl, —O-alkyl-, amino-, alkylamino- or dialkylamino groups, halogen atoms, —OH, CN, NO₂, S, —SR, O and C(O), whereby R means a C₁-C₆ alkyl group such as methyl, ethyl, butyl, n-propyl, iso-propyl or hexyl or a C₅-C₁₂ aryl group such as phenyl or naphthyl

In the above-indicated formulae of the compounds according to the invention and in the following “alkenyl” represents a linear or branched alkenyl group, particularly C₂-C₈ alkenyles with one or several double bonds as ethene, propene, butene, etc. A substituted alkenyl is an alkenyl substituted by aryl, heteroaryl, halogen atoms, CN, NO₂, S, —SR, O and C(O), whereby R means a C₁-C₆ alkyl group such as methyl, ethyl, butyl, n-propyl, iso-propyl or hexyl or a C₅-C₁₂ aryl group such as phenyl or naphthyl

In the above-indicated formulae of the compounds according to the invention and in the following “alkynyl” represents a linear or branched alkynyl group, particularly C₂-C₈ alkynyles with one or several triple bonds as ethynyl, propynyl, butynyl, etc. A substituted alkynyl is an alkynyl substituted by aryl, heteroaryl, halogen atoms, CN, NO₂, S, —SR, O and C(O), whereby R means a C₁-C₆ alkyl group such as methyl, ethyl, butyl, n-propyl, iso-propyl or hexyl or a C₅-C₁₂ aryl group such as phenyl or naphthyl

Alkylamino represents a C₁-C₁₂ alkyl substituted amino group, such as methylamino, ethylamino or propylamino.

Dialkylamino represents a amino group wherein two hydrogen atoms have been substituted by C₁-C₁₂ alkyl groups, such as dimethylamino, diethylamino, or methylethylamino.

Acyl groups comprise linear or branched acid radicals (—R—CO—), wherein R is a C₁-C₆ alkyl group, such as methanoyl, ethanoyl, or propanoyl.

Multiple-membered aromatic ring systems are C₃-C₁₃ cyclic systems which can be unsaturated or saturated and can be substituted by alkyl and aryl and in the case of heteroaromatic ring systems by heteroaryl, halogen, CN, NO₂, S, O, C(O).

Aryl is defined as a organic radical which can have resulted from arenes, i.e. any mono or polycyclic aromatic and heteroaromatic hydrocarbon compounds after cleavage of a hydrogen atom; aryl is particularly phenyl or naphthyl. Heteroaryl is particularly five- to six-membered aromatic compounds containing nitrogen, oxygen or sulphur. Aryls and heteroaryls can be an aryl substituted by alkyl, aryl, heteroaryl, halogen atoms, CN, NO₂ and C(O). Particularly preferred examples of hetroaryl are pyridyl, pyrrolidine and furanyl.

Halogen is F, Cl, Br and I.

If pharmaceutically acceptable salts of the compounds according to the invention are used, those salts are preferably derived from inorganic or organic acids or bases. Included among such acid salts are the following: acetate, adipate, alginate, aspartate, bezoate, benzene sulfonate, bisulfonate, butyrate, citrate, camphorate, camphor sulfonate, cyclopentane propionate, digluconate, dodecylsulfate, ethanesulfonate, fumarate, glucoheptanoat, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.

Base salts include ammonium salts, alkali metal salts, such as sodium and potassium salts, alkaline earth salts, such as magnesium and calcium salts, salts with organic bases, such as dicylohexamine salts, N-methyl-D-glucamine, and salts with amino acids such as arginine, lysine and so forth. Also the basic nitrogen containing groups may be quaternized with agents such as lower alkyl halides, such as methyl, ethyl, propyl, and butyl chlorides, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides such as benzyl and phenethyl bromides and other.

As explained above, it is supposed, without being bound to a certain theory, that the advantageous properties of the compound according to the invention are due to the fact that it has an affinity to (PO₃H₂)Ser/(PO₃H₂)Thr-Pro-specific peptidyl-prolyl-cis/trans-isomerases of the parvuline type, such as the human Pin1 and that it is particularly able to inhibit their peptidyl-prolyl cis/trans-isomerase activity. How this inhibitory effect of the compounds according to the invention can be determined has already been explained above and has been shown exemplary in Example 1.

Another aspect of the invention provides methods for the therapy of diseases which are based on the use of inhibitors for PPlases of the parvuline type. PPlases of the parvuline family have been identified in the most varied organisms. For eucaryotic representatives as the human Pin1 and the ESS1/PTF1 from yeast, an essential participation in mitotic processes has been shown. Thus, the compounds according to the invention can be used for the treatment of a large number of functional disorders in cell proliferation and mitosis.

Cell proliferative functional disorders which can be treated with the above-identified compounds and methods are disorders with abnormal, uncontrolled and unwanted cell growth. Some examples are: cancer, fibrosing diseases, non-neoplastic changes as e.g. prostate hypertrophy, endometriosis, psoriasis and others. Among the treatable cancers are hematopoetic changes as leukemias, lymphomas and also carcinomas, sarcomas, osteomas, fibrosarcomas, chondrosarcomas and others. Specific cancers which are intended for the treatment with the active compound according to the invention are breast cancer, prostate cancer, cervical carcinoma, gastric carcinoma, bladder carcinoma, cancer tumours, lung cancer, colon cancer, pancreatic carcinoma, liver carcinoma, renal carcinoma, and others. The indicated fibrosing diseases include functional disorders as fibromyalgia, fibroses (cystic, hepatic, pulmonary, pericardial and others), fibromuscular hyperplasia, restenosis, arteriosclerosis and similar diseases.

The compounds according to the invention can additionally be used for the treatment of infectious diseases which are caused by viruses, bacteria and fungi, as well as for the treatment of diseases which are caused by parasitic protozoa. Among the viral infections which can be treated with the compound according to the invention, its derivatives and with the methods according to the invention are infections caused by RNA and DNA viruses, as e.g. adenoviruses, arboviruses, arenaviruses, Bunyaviruses, dengue viruses, flaviviruses, herpes viruses, paramyxoviruses, picomaviruses, polyomaviruses, Orbiviruses, orthomyxoviruses, rhabdoviruses, retroviruses, rubella viruses, togaviruses and others. Among these diseases are AIDS, hepatitis, encephalitis, meningitis, hemorrhagic fever, colds, hepatitis, Bluetongue, Colorado tick fever, Lassa fever and border disease.

The bacterial infections which can be treated with the compounds according to the invention and the methods include infections caused by gram-positive or gram-negative bacteria as well as by Bacillus, Camphylobacter, Clostridium, Diplococcus, Enterobacter, Enterococcus, Erysipelothricosis, Escherichia, Hemophilus, Klebsiella, Listeria, Morganella, Mycobacterium, Neisseria, Proteus, Providencia, Salmonella, Serratia, Shigella, Staphylococcus, Streptococcus, Yersinia and others. The cited microorganisms cause diseases as e.g. brucellosis, cholera, diarrhea, gastroenteritis, gonorrhea, Lyme disease, mastoiditis, meningitis, anthrax, pneumonia, rheumatic fever, dysentery, tetanus, tuberculosis, enteric fever, etc. Among the fungal infections which can be treated with the compounds according to the invention and the methods according to the invention are fungal infections which either affect the whole organism, the skin or the urogenital tract. The infections which affect the whole body are caused by: Absidia, Aspergillus, Candida, Coccidioides, Cryptococcus, Blastocyces, Histoplasma, Hormodendrum, Mucor, Nocardia, Paracoccidioides, Phialopora, Rhinosporidium Rhizopus, Sporothrix, etc. Infections of the skin are caused by fungi as, e.g. Microsporum, Trichophyton, Epidermophyton, Candida and Pityrosporum. Fungal infections affecting the urogenital tract are caused by fungi as Aspergillus, Candida, Cryptococcus and Zygomycodoides. All of these fungi moreover cause infectious diseases as e.g. epidermophytosis, San Joaquin Valley fever. These diseases can have severe and fatal consequences especially in the case of patients with immunodeficiency, as e.g. after organ transplantations or with AIDS (Acquired Immunodeficiency Syndrome).

The compounds according to the invention and the indicated methods can also be used for the treatment of parasitic diseases. Causes for these diseases are parasitic human protozoa as Trypanosoma, Leishmania, Trichomonas, Giardia, Entamoeba, Plasmodium, Toxoplasma and Balantidium. Representatives of the afore-mentioned species cause diseases as e.g. the sleeping disease, the Chagas' disease, trichomoniasis, forms of dysentery, malaria and toxoplasmosis. The compounds according to the invention and the indicated methods can also be used in the treatment of autoimmune diseases as e.g. psoriasis, neurodermatitis, systemic lupus erythematosus, glomerulonephritis, multiple sclerosis, Basedow's disease, chronic thyroiditis, myasthenia gravis, pemphigus, dermatosclerosis, ulcerative colitis, rheumatoid arthritis, purpura, hemolytic anemia, diabetes mellitus type I, uveitis, Cogan's syndrome, etc.

In another aspect the compounds according to the invention can be used as pesticides, in the form of insecticides and herbicides. The compounds according to the invention blocks the mitosis in insect and plant cells. The compounds according to the invention can thus be used in the growth and proliferation control of animal and plant parasites. Insecticides are biologically active compositions which are effective against insects and their forms of development. The most important areas of application are hygiene (insecticides against active or passive carriers and intermediate hosts of diseases and epidemics as flies, gnats, fleas and bugs in the case of humans and animals), the protection of plants and stocks. Herbicides are chemical weed killers. Weed and useful plants compete for light, water, nutrients and living space. By using herbicides, weed can be reduced and thus, yields can be increased.

The present invention moreover provides the use of the compounds according to the invention and pharmaceutical compositions containing the compounds according to the invention optionally in combination with a pharmaceutically acceptable carrier or binder. In the course of a therapy, a therapeutically relevant dose of the compound according to the invention should be administered. This is a specific amount of the compound according to the invention which, if administered, leads to an improvement of the symptoms and to the increase in the patient's lifespan. The toxicity and the therapeutically efficient dose of the compound according to the invention can be determined in the form of LD₅₀ and ED₅₀ values in cell culture assays and animal tests (Application Example 2). The LD₅₀ value describes the dose leading to a lethality of 50% in a population, while the ED₅₀ value describes the dose which is therapeutically efficient in 50% of a population. The ratio between toxicity and therapeutic effect is expressed in the quotient LD₅₀/ED₅₀. In this connection, naturally, compounds with a high therapeutic potential are preferred. The data obtained by the cell culture assays and the animal tests are subsequently used as the basis for determining the doses for the treatment of humans. These are preferably in the range of the ED₅₀ value found if it is slightly or not at all toxic. The dose administered varies depending on different factors, as e.g. the form of administration of the substance, the kind of administration, the state of health of the patient and others and lies within the discretion of the physician. In order to formulate the necessary dose more exactly, for each of the compounds according to the invention the therapeuctically effective dose can be determined by additionally determining the IC₅₀ value. This is done as was already described in the introduction and as shown in example 1. Thus, starting from this value, by means of animal tests, a dose present in the circulating plasma of the organism can be formulated which is within a concentration range which also corresponds to the IC₅₀ value of the compound in the cell culture assay. The treating physician will not only determine the corresponding dose for treatment but naturally also when the treatment is ended, interrupted or when the dose has to be reduced due to toxicity, malfunctions of organs and others. It is also possible for the physician to order an increase in the dose, taking into consideration all factors mentioned, if the desired therapeutic effects do not materialise. The amount of the prescribed dose and the duration of the treatment vary depending on different factors, e.g. the form of administration, the kind and intensity of the disease. The dose and the duration of the treatment also depend on the body weight, the age, sex and the reaction of every individual patient to the medicament. Typically, the dose administered is between 1 to 50 mg/day/kg body weight. 1 to 50 mg should be administered to a child and between 25 and 1,000 mg should be administered to an adult per day. The compounds according to the invention according to the formulae (1), (2), (3), (4), (5), (6), (7) und (8) as well as its pharmaceutically acceptable salts according to the invention can be administered systemically or topically. Methods and techniques for the formulation and the administration can be taken from “Remington's Pharmaceutical Sciences” [26]. Known methods for the administration are oral, rectal, vaginal, intestinal or by application onto the skin or the mucous membranes. Possible routes with respect to a parenteral uptake are intramuscular and subcutaneous injections and intrathecal, intraventricular, intravenous, intraperitoneal, intranasal and intraocular injections. The formulation of the compounds according to the invention can be effected as a solution (preferably for parenteral administration) or bound to carriers (e.g. for oral application) which are well-known to the person skilled in the art. Through coupling to specific carriers it is possible to administer the substance in the form of tablets, pills, capsules, coated tablets, liquids, gel, granulate, sirup, suspensions, slurry, etc. For parenteral uptake, the compounds should be formulated in an aqueous solution, preferably in physiological buffers as Hank's buffer, Ringer's solution or PBS (phosphate buffered saline) buffer. For transmucosal injections, penetration agents which are commonly used and which are known to the person skilled in the art can be used. Apart from buffer solutions and water, emulsions such as e.g. oil-in-water emulsions can also be used. In this connection, suitable lipophylic solvents and substances are e.g. fatty oils like sesame oil, synthetic fatty acid esters as ethyloleate or triglycerides or liposomes. Suspensions for injection can also contain components which increase the viscosity of the suspension, e.g. sodium carboxymethyl cellulose, sorbitol, dextran and others. For the manufacture of highly concentrated solutions, the solubility of the substance according to the invention can optionally be increased by stabilisers and reagents which are generally known to the person skilled in the art. If the uptake of the substance according to the invention is to be effected directly into the cell, if possible, this can be done with the help of liposomes. Lipsosomes are spherical lipid layers surrounding a hydrophilic lumen in which the substance according to the invention is enclosed. The encapsulated content of the liposomes is thus protected against environmental influences and can thus be efficiently transferred into the cell after the fusion of the lipid layer of the liposomes with the cell membrane of the eukaryotic cell. The mode of action of these liposomal systems is discussed in the published patents International Patent Publication No. WO 91/02805 and International Patent Publication No. WO 91/19501 and in U.S. Pat. No. 4,880,635. Suitable pharmaceutical carriers for oral administration of the substances according to the invention are also known to the person skilled in the art and comprise filling components as e.g. lactose, sucrose, mannitol, sorbitol, cellulose components such as corn, wheat, rice and potato starch, gelatine, methylcellulose, hydroxypropylmethyl cellulose, sodiumcarboxymethylcellulose, polyvinylpyrrolidone and others, as well as mixtures of the cited substances. Accordingly, the carrier or the diluent can contain known substances for the delayed release of the substance according to the invention as glycerolmonostearate or glyceroldistearate alone or in combination with a wax. If necessary, autolytic substances as agar, cross-linked polyvinylpyrrolidone, alginic acid, etc. or their salts can be used. If the compounds according to the invention is to be administered in the form of a coated tablet, the concentrated sugar solutions which e.g. contain gum arabicum, talc, polyvinylpyrrolidone, polyethyleneglycol, titanium dioxide, suitable organic solvents or their mixtures and others and which are usually used by the person skilled in the art can be used for coating the coated tablets. Food colourings and pigments can be added to colour the coated tablets and capsules and in order to be able to better distinguish them as well as to mark different doses. In the following, the invention is described in detail.

Synthesis of the Compounds of the Invention

The production of the compounds according to the invention was carried out according to the following reaction schemes 1 to 7. 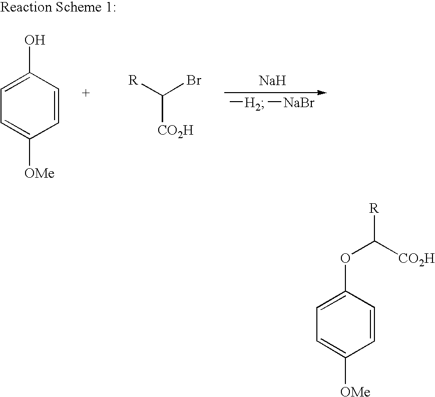
In a 1 l-three-necked flask with reflux condenser, internal thermometer, nitrogen/vacuum attachment and a septum 4.64 g sodium hydride (60% in liquid paraffin; Fluka) (approx. 1.15 mol) were weighed in. By evacuation and ventilation with nitrogen, a nitrogen atmosphere was produced in the flask. While stirring, 10 ml abs. hexane was added, the stirring was interrupted and the supernatant solvent was removed with a syringe. This process was repeated once more. Subsequently, an evacuation was carried out to remove the rests of the solvent. 210 ml abs. tetrahydrofuran (THF) were added to the remaining sodium hydride which was freed from mineral oil. The suspension was cooled in an ice bath while stirring. With the help of the dropping funnel, a solution of 5.77 g (46.5 mmol) 4-hydroxyanisole in 60 ml THF was added drop by drop so as not to raise the internal temperature above 5° C. Then the suspension was heated for 1 hour under reflux, was stirred over night at room temperature and was subsequently once more cooled in an ice bath. Then a solution of 10.0 g (46.5 mmol) α-bromophenylacetic acid in 35 ml abs. THF was added with the dropping funnel so as not to raise the temperature over 5° C. After heating for 6 hours to 60° C. and subsequent cooling down to room temperature, the mixture was poured onto 1.5 l ice. After acidification with 1N hydrochloric acid to pH=2, five extractions with a total of 300 ml diethyl ether were carried out. The combined organic phases were dried over magnesium sulfate, the solvent was removed in the rotary evaporator and the residue was recrystallised from n-hexane/ethyl acetate (1:1). Thus, 7.5 g (62%) α-(4-methoxyphenoxy)phenylacetic acid (R═C₆H₅) were obtained. An alternative formula according to Camps et al., 1997 is known in the literature [27]. Analogously, (4-methoxyphenoxy)acetic acid (R═H) was obtained from 4-hydroxyanisole and bromoacetic acid; yield after recrystallisation from chloroform: 66%; cf. [28].

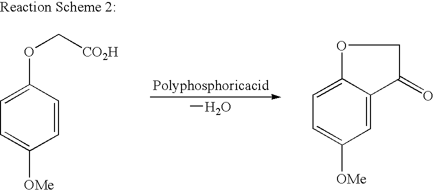

In an 1 l-three-necked flask with a drying pipe, KPG stirrer and glass stopper, 310 g polyphosphoric acid (84%; Across Organics) were weighed in and were heated at an oil bath temperature of 75° C. Within 10 min 8.6 g (47.15 mmol) (4-methoxyphenoxy)acetic acid were added; then stirring was carried out for 40 min at 75° C. The cooled solution was poured onto 1.5 l ice. After stirring during 2 hours, three extractions were carried out with a total of 400 ml chloroform. The combined organic phases were washed with water, 10% K₂CO₃ solution and again with water and were dried over Na₂SO₄. After removal of the solvent in the rotation evaporator, the residue was purified by column chromatography (silica gel 60; chloroform). Thus, 774 mg (10%) 5-methoxy-3(2H)-benzofuranone were obtained. For an alternative formula, cf. Hammond et al.; 1990 [29].

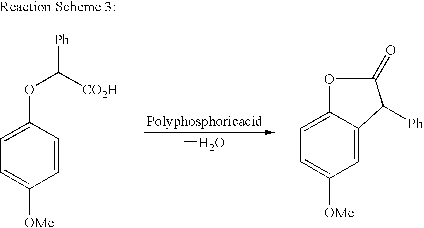

Under conditions analogous to those in equation 2, a yield of 40% 5-methoxy-3-phenyl-2(3H)-benzofuranone was obtained from α-(4-methoxyphenoxy)phenylacetic acid by rearrangement; cf. Khosla et al. [30].

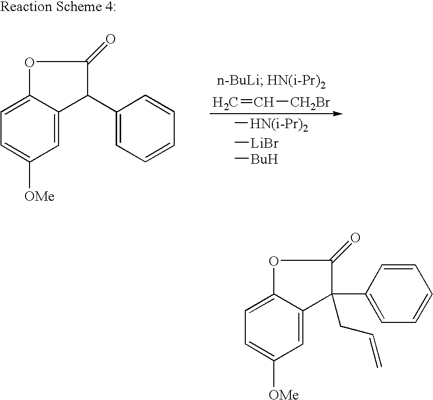

Under nitrogen atmosphere, 2.07 ml (14.74 mmol) diisopropylamine were solved in a 250 ml-round flask with internal thermometer and attachment to the nitrogen/vacuum line in 52 ml abs. THF and were cooled to −78° C. While stirring 9.75 ml (15.6 mmol) n-butyllithium (1.6 M solution in n-hexane; Acros Organics) were added drop by drop so as not to raise the internal temperature over −70° C. Subsequently, the solution was stirred for 30 min at 0° C. In a 50 ml-round flask with nitrogen/vacuum attachment, 2.95 g (12.3 mmol) 5-methoxy-3-phenyl-2(3H)-benzofuranone were solved under nitrogen atmosphere in 20 ml abs. THF. This mixture was added drop by drop via a cannula to the solution of lithiumdiisoproplyamide which was cooled down to −78° C., with the 250 ml-round flask being slightly evacuated. During the addition, the temperature was retained at below −70° C. After stirring for 2 hours at −78° C., 1.24 ml (14.74 mmol) allyl bromide and 1.78 ml (14.74 mmol) 1,3-dimethyltetrahydro-2(1H)-pyrimidinone were added drop by drop to the enolate solution. Within 4 days, the solution was allowed to heat to room temperature. The organic phase was washed with sat. NH₄Cl solution and the aqueous phase was extracted three times with chloroform. The combined organic phases were dried over Na₂SO₄. After removal the solvent in the rotary evaporator and purification by column chromatography [silica gel 60; n-hexane/toluene/ethyl acetate (5:3:2)] 5-methoxy-3-phenyl-3-(2-propenyl)-2(3H)benzofuranone was obtained; yield: 3.08 g (89%); R_{f}=0.79. 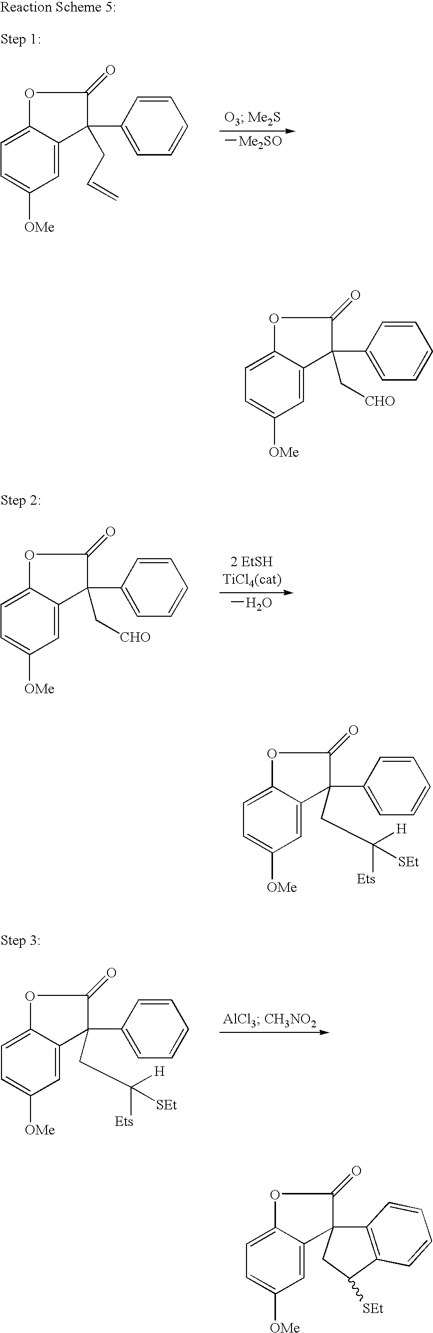 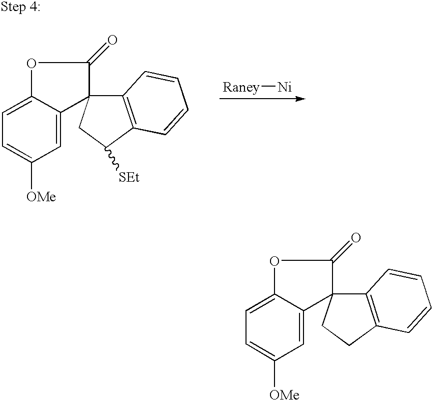
Step 1:

In a solution of 2.83 g (10.1 mmol) 5-methoxy-3-phenyl-3-(2-propenyl)-2(3H)-benzofuranone in 50 ml abs. dichloromethane at 78° C., ozone was introduced via a frit until a light bluish colouring remained: excessive ozone was removed at the same temperature with an influx of oxygen and then with nitrogen. After adding 50 mmol dimethyl sulfide (for synthesis; Merck), the solution was allowed to heat to room temperature within 2 hours. The organic phase was washed with water several times and was dried with Na₂NO₄. After removing the solvent with the rotary evaporator, the residue obtained was purified by column chromatography (silica gel 60: running substance: acetic ester, R_{f}=0.9); yield in aldehyde: 1.48 g (52%).

Step 2:

A solution of the aldehyde obtained in step 1 (1.48 g, 5.24 mmol) and 0.82 g (13.15 mmol) ethanethiol (Merck) were cooled down to −10° C. to −15° C. in 5 ml abs. chloroform. While stirring 0.07 ml (0.67 mmol) titanium tetrachloride were added drop by drop. Within a few minutes the reaction temperature was allowed to heat up to 25° C. while stirring and the stirring was continued for 1 hour at room temperature. After the termination of the reaction, water was added to the solution. The organic phase was separated, the aqueous phase was extracted with 100 ml chloroform. The combined organic phases were washed with water and with saturated sodium chloride solution and were dried over Na₂SO₄. Then the solvent was removed in a rotary evaporator. The thioacetal (2.04 g, quantitative) thus obtained was further reacted as a raw product without purification in step 3.

Step 3:

A solution of 2.04 g (5.24 mmol) of the raw product obtained in step 2 in 20 ml abs. nitromethane was added drop by drop to a solution of 2.02 g aluminium trichloride (sublimated, powdered, for synthesis; Merck) which was stirred in an inert gaseous atmosphere at 0° C. The solution was allowed to heat up to room temperature over night. For processing, approx. 2 ml 1 N hydrochloric acid were added and the largest portion of the solvent was removed under vacuum. The residue was taken up in dichloromethane, washed twice with water in the separating funnel and was dried after the separation of the phases over Na₂SO₄. The raw product after removal of the solvent in the rotary evaporator (yield: 1.57 g, 92%) was processed without purification in step 4.

Step 4:

In a 250 ml one-necked flask, 1.57 g (4.82 mmol) of the raw product obtained in step 3 were stirred with 36 g Raney-Nickel (10% suspension in water; Fluka) in 200 ml abs. methanol for 6 days at room temperature. The solution was filtered, the residue was taken up in methanol and was filtered via a small (dry) column filled with silica gel so as to remove residues of metals and metal salts. The solvent was removed in the rotary evaporator, the remaining raw product was purified with a column chromatography. [Silica gel 60; n-hexane/acetic ester (2:1)]; R_{f}=0.73. Thus, 1′,2′,3′H-spiro[indene-3′,3-(3H-5-methoxy-benzofurane-2-one)] was obtained; yield 0.154 g (12%).

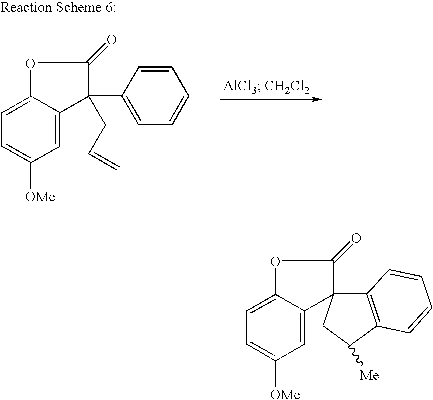

A solution of 5-methoxy-3-phenyl-3-(2-propenyl)-2(3H)-benzofuranone (50.0 mg, 0.18 mmol) in 5 ml abs. dichloromethane was added drop by drop to a solution of 36 mg (0.27 mmol) aluminium trichloride in 5 ml abs. dichloromethane which was stirred under nitrogen atmosphere at 0° C. The solution was allowed to heat up to room temperature over night while stirring. After adding approx. 2 ml 1 N hydrochloric acid the mixture was taken up in 100 ml chloroform, was washed twice in the separating funnel and was dried over Na₂SO₄. The raw product which was obtained after removing the solvent under vacuum was dried by column chromatography. [Silica gel 60; n-hexane/chloroform (1:5)]. R_{f}=0.49). Yield in 1′,2′,3′H-spiro[1′-methyl-indene-3′,3-(3H-5-methoxy-benzofurane-2-one)]: 11.8 mg, (24%).

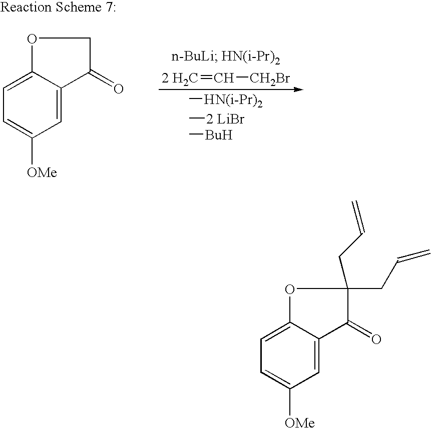

As explained in the formula pertaining to equation 3, 56.4 mg (0.34 mmol) 5-methoxy-3(2H)-benzofuranone were processed with 1.4 equivalents allyl bromide. Thus, the following was obtained: 5-methoxy-2,2-di-(2-propenyl)-3(2H)-benzofuranone; yield after purification by column chromatography [silica gel 60; acetic ester]: 30 mg (36%); R_{f}=0.93.

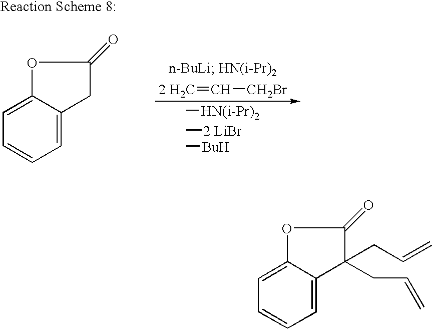

As described in the formula of equation 3, 178.4 mg (1.33 mmol) 2(3H)-benzofuranone were reacted with 1.4 equivalents allyl bromide. Thus, the following was obtained: 3,3-di-(2-propenyl)-2(3H)-benzofuranone; yield after purification with column chromatography [(silica gel 60; n-hexane/toluene/acetic acid (5:3:2)]: 187.2 mg (65.7%); R_{f}=0.88.

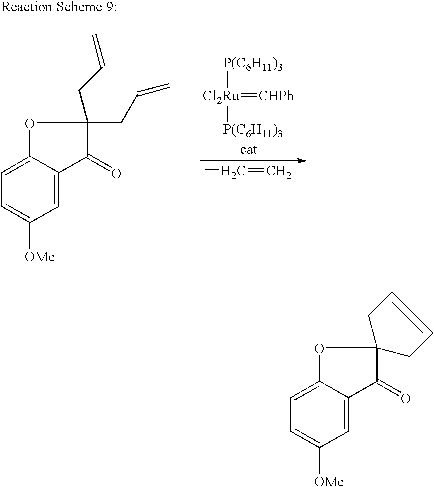

30 mg (0.12 mmol) 5-methoxy-2,2-di-(2-propenyl)-3(2H)-benzofuranone were solved in 6 ml abs. dichloromethane under nitrogen atmosphere and were added at 20° C. to a solution of 20 mg (0.02 mmol) benzylidene-bis(tricyclohexylphosphine)dichlororuthenium (purum, >97%; Fluka) in 50 ml absolute dichloromethane. The mixture was stirred for 19 hours at room temperature and then the solvent was removed under vacuum. The purification of the remaining raw product by column chromatography [silica gel 60; chloroform] yielded 17.5 mg (66%) 1′,2′,3′ H-spiro[cyclopentene-3′,2-(2H)-5-methoxy-benzofurane-3-one](R═H); R_{f}=0.71.

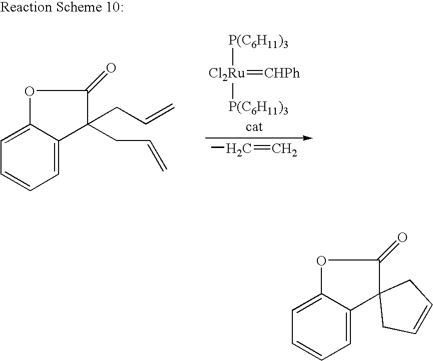

The analogous reaction of 3,3-di-(2-propenyl)2(3H)-benzofuranone (844 mg, 3.94 mmol) yielded 1′,2′,3′H-spiro[cyclopentene-3′,3-(3H)-benzofurane-2-one] after purification by column chromatography [silica gel 60; chloroform]; yield: 619 mg (85%); R_{f}=0.81.

### EXAMPLE 1

### Determining the Inhibition Constants of the Derivatives of the Substance According to the Invention

Determining the inhibition constants of the selected compound according to the invention 1′,2′,3′H-spiro[indene-3′,3-(3H-5-methoxy-benzofurane-2-one)] vis-à-vis the PPlase hPinl by protease-coupled PPlase assay:
Buffer: 35 mM HEPES (pH 7.8), 1,200 μl
Substrate: Ac-Ala-Ala-(PO₃H₂)Ser-Pro-Arg-(4-)nitroanilide stock solution 10 mg/ml in 35 mM Hepes (pH 7.8),
Auxiliary protease: trypsin (Roth), stock solution 10 mg/ml in 35 mM Hepes (ph 7.8), concentration in the solution for measuring 0.34 mg/ml
Enzyme: human Pin1 (recombinant from E. coli), stock solution 0.55 μM, concentration in the solution for measuring: 2 nM
Effectors: effector stock solution 10 mM in DMSO, concentration in the solution for measuring between 1,000 and 0.01 μM
Temperature: 10° C.

In the incubation solution, the effectors were preincubated with the enzyme for 5 min, then 3.5 μl of the auxiliary protease trypsin were added and the reaction was started immediately afterwards by adding 3.5 μl substrate. The reaction was monitored at a wave length of 390 nm with the measuring instrument Hewlett Packard UVNIS spectrophotometer HP 8452A. The kinetic analysis of the data was carried out by “SigmaPlot” (Scientific Graphing System Vers. 2.0, Jandel Corp.).

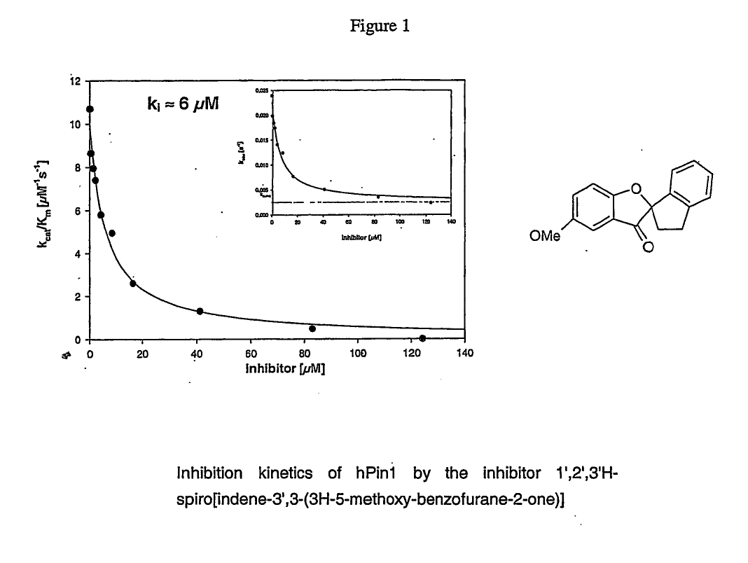

FIG. 1 summarizes exemplarily the inhibition kinetics for 1′,2′,3′H-spiro[indene-3′,3-(3H-5-methoxy-benzofurane-2-one)].

In Table I, typical inhibition constants for compounds according to the invention are summarised.

TABLE 1

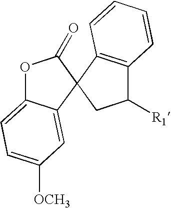

Formula A

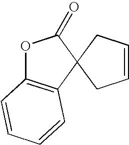

Formula B

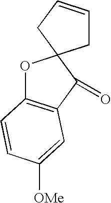

Formula C

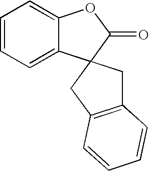

Formula D

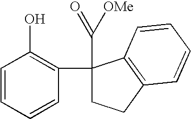

Formula E

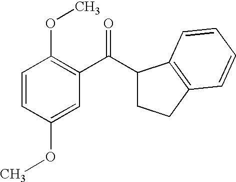

Formula F

| | Basic structure | hPin1- |
|---|---|---|
| Compound | according to formula | inhibition K₁[μM] |
| 1′,2′,3′H-spiro[indene-3′,3- | A | 6 |
| (3H-5-methoxy- | (with R₁ = —H) | |
| 1′,2′,3′H-sprio[1′-methyl- | A | 20 |
| indene-3′,3-(3H-5- | (with R₁ = —CH₃) | |
| methoxy-benzofurane-2- | | |
| one)] | | |
| 1′,2′,3′H- | B | 150 |
| spiro[cyclopentene-3′,3- | | |
| (3H-benzofurane-2-one)] | | |
| 1′,2′,3′H- | C | 65 |
| spiro[cyclopentene-3′,2- | | |
| (2H-5-methoxy- | | |
| benzofurane-3-one)] | | |
| 1′,2′,3′H-sprio[indene-2′,3- | D | 7 |
| (3H-benzofurane-2-one)] | | |
| 2-hydroxyphenyl-(1- | E | 27 |
| methoxy-indane-1-yl)- | | |
| ketone | | |
| 1-(2,5-dimethoxyphenyl)- | F | 50 |
| 1′-indane-1′-1-methanone | | |

### EXAMPLE 2

### Measuring the Cytotoxicity of the Compounds According to the Invention

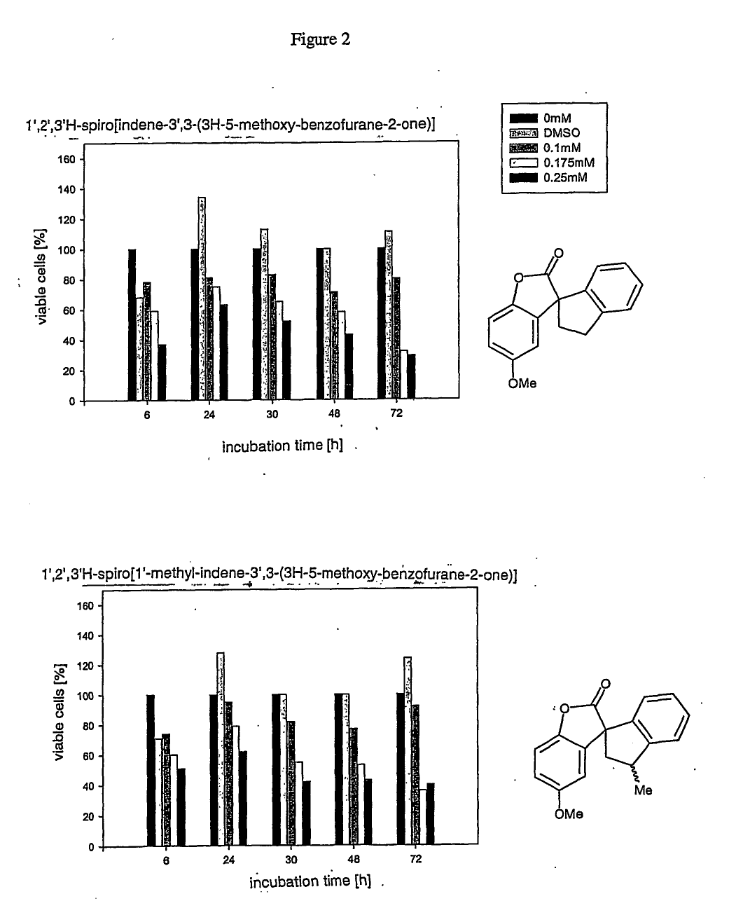

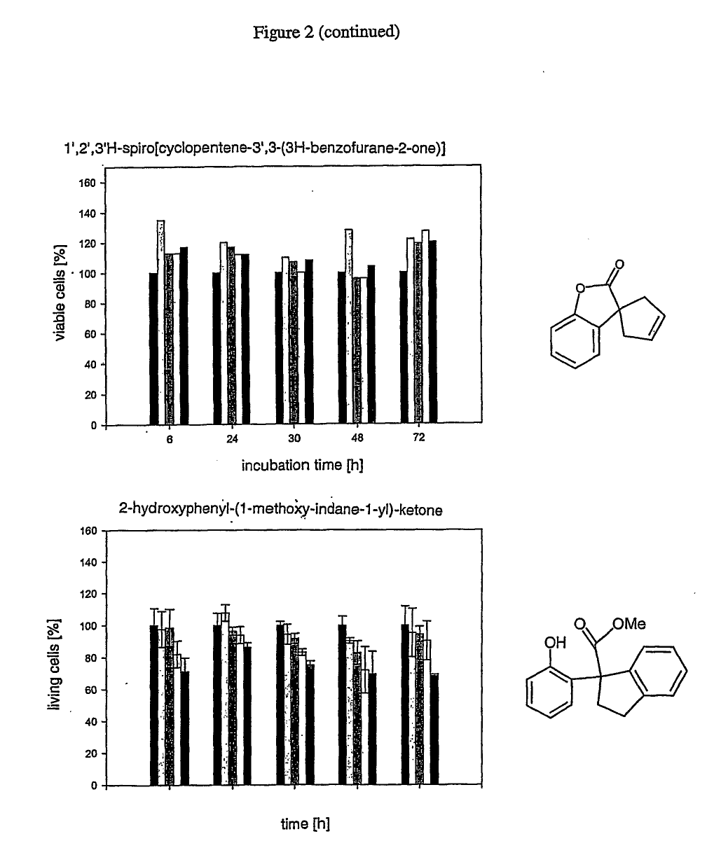

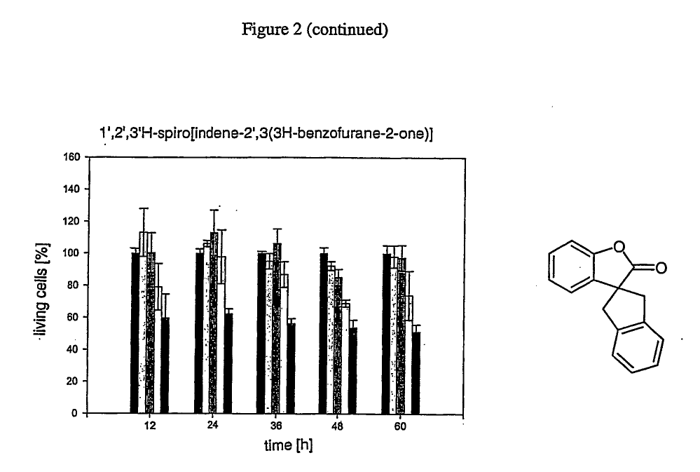

Determining the cytotoxicity of the compounds according to the invention was carried out with an MTT-test according to Mosmann (1983) [31]. Hereby, the tetrazolium salt MTT [3-(4,5-dimethylthiazole-2-yl)-2,5-diphenyltetrazoliumbromide] is used in a quantitative assay for determining the survival and proliferation rate of mammalian cells. With the help of the assay, living cells were detected. MTT itself is a yellowish-brown salt which, if incubated with living cells having active mitochondria, is cleaved in a deeply blue formazan product. Subsequently, the reaction carried out can be measured with a spectrophotometer and analysed. The vitality rate of the cells is directly proportional to the absorption of the blue dye at 550 nm. In the present test 5,000 Hela cells per well were seeded in a 96-well plate and were cultured over night for attachment in DMEM (5% FCS, glutamine, antibiotics). Then, fresh medium was added with the corresponding concentration of the corresponding substance according to the invention which was dissolved in DMSO (0 mM; 0.1 mM, 0.175 mM or as control the corresponding amount of DMSO in DMEM with 5% FCS and glutamine). After 6 h, 24 h, 30 h, 48 h or 72 h in each case the rate of viable cells was determined. For this, 2.4 mg MTT in 10 ml serum-free DMEM were solved for 30 min at 37° C. Subsequently, the cell culture medium of the cells in the microtiter plate was removed and 100 μl of the MTT medium solution were added per well. After another incubation for 1 hour at 37° C., the medium was removed and 200 μl DMSO were added in each case. The microtiter plate was thoroughly shaken to solve the cells completely in DMSO. The rate of viable cells was then determined by measuring the absorption at 550 nm and 630 nm with a MR7000 (Dynatech). The analysis was carried out by determining the absorption difference (ΔAbs=Abs₅₅₀ₙₘ−Abs₆₃₀ₙₘ). FIG. 2 shows the corresponding average values of three independent measuring points. In FIG. 2, the values which were obtained for the untreated cells correspond to 100%.

### LITERATURE

1 Fischer G, Mech C, Bang H: Nachweis einer Enzymkatalyse für die cis/trans Isomerisierung der Peptidbindung in prolinhaltigen Peptiden. Biomed Biochim Acta 1984,43: 1101-1111.
2 Fischer G: Peptidyl-prolyl cis/trans isomerases and their effectors. Angew. Chem Int Ed 1994, 33:1415-1436.
3 Galat A., Metcalfe S M: Peptidylproline cis/trans isomerases, Prog Biophys Molec Biol 1995, 63: 67-118.
4 Kay J E: Structure-function relationships in the Fk506-binding protein (Fkbp) family of peptidylprolyl cis-trans isomerases. Biochem J 1996, 314: 361-385
5 Hennig L, Christner C, Kipping M, Schelbert B, Rücknagel K P, Grabley S, Kullertz G, Fischer G: Selective inactivation of parvulin-like peptidyl-prolyl cis/trans isomerases by juglone. Biochemistry 1998, 37:5953-5960.
6 Monks T J, Hanzlik R P, Cohen G M, Ross D, Graham D G: Quinone chemistry and toxicity. Toxicol Appl Pharmacol 1992, 112:2-16.
7 Didry N, Dubreuil L, Pinkas M: Activity of anthraquinonic and naphthoquinonic compounds on oral bacteria. Pharmazie 1994, 49:681-683.

8 Lu K P, Hanes S D, Hunter T: A human peptidyl-prolyl isomerase essential for regulation of mitosis. Nature 1996, 380: 544-547.
9 Hanes S D, Shank P R, Bostian K A: Sequence and mutational analysis of ESS1, a gene essential for growth in Saccharomyces cerevisiae. Yeast 1989, 5:55-72.
10 Hani J, Stumpf G, Domdey H: PTF1 encodes an essential protein in Saccharomyces cerevisiae, which shows strong homology with a new putative family of PPlases. FEBS Left 1995, 365: 198-202.
11 Nigg E A: Cyclin-dependent protein kinases: key regulators of the eukaryotic cell cycle. Bioessays 1995, 17:471-480.
12 Lu P J, Wulf G, Zhou X Z, Davies P, Lu K P: The prolyl isomerase Pin1 restores the function of Alzheimer-associated phosphorylated tau protein [see comments]. Nature 1999, 399:784-788.
13 Shen M H, Stukenberg P T, Kirschner M W, Lu K P: The Essential Mitotic Peptidyl-Prolyl Isomerase Pin1 Binds and Regulates Mitosis-Specific Phosphoproteins. Genes & Dev 1998, 12: 706-720.
14 Albert A, Lavoie S, Vincent M: A hyperphosphorylated form of RNA polymerase II is the major interphase antigen of the phosphoprotein antibody MPM-2 and interacts with the peptidyl-prolyl isomerase Pin1. J Cell Sci 1999, 112:2493-2500.
15 Winkler K E, Swenson K I, Kornbluth S. Means A R: Requirement of the prolyl isomerase Pin1 for the replication checkpoint. Science 2000, 287: 1644-1647.
16 Wu X., Wilcox CB, Devanshayam G., Hackett R L, Arevalo-Rodriguez M, Cardenas M E, Heitman J. Hanes S D: The Ess1 prolyl isomerase is linked to chromatin remodelling complexes and the general transcription machinery. Embo J 2000,19:3727-3738.
17 Ryo A, Nakamura M, Wulf G, Liou Y C, Lu K P: Pin1 regulates turnover and subcellular localization of beta-catenin by inhibiting its interaction with APC. Nat Cell Biol 2001, 3: 793-801.
18 Wulf G M, Ryo A, Wulf G G, Lee S W, Niu T, Petkova V, Lu K P: Pin1 is overexpressed in breast cancer and cooperates with Ras signalling in increasing the transcriptional activity of c-Jun towards cyclin D1. Embo J 2001, 20: 3459-3472.
19 Zhou X Z, Kops O, Werner A, Lu P, Shen M, Stoller G, Kullertz G, Stark M, Fischer G, Lu K P: Pin1-Dependent Prolyl Isomerization Regulates Dephosphorylation of Cdc25C and Tau Proteins. Mol Cell 2000, 6: 873-883.
20 Küllertz G, Lüthe S. Fischer G: Semi-automated microtiter plate assay for monitoring peptidyl-prolyl-cis/trans isomerase activity in normal and pathological human sera. Clin Chem 1988, 44: 502-508.
21 Janowski B. Wollner S. Schutkowski M, Fischer G: A protease-free assay for peptidyl-prolyl cis/trans isomerases using standard peptide substrates. Analyt Biochem 1997, 252: 299-307.
22 Brandsch M, Thunecke F, Kullertz G, Schutkowski M, Fischer G, Neubert K: Evidence for the absolute conformational specificity of the instestinal H⁺/peptide symporter, PEPT1. J Biol Chem 1998, 273: 3861-3864
23 Scholz C, Rahfeld J, Fischer G, Schmid F X: Catalysis of protein folding by parvulin. J Molec Biol 1997, 273: 752-762
24 Kern D, Kern G, Scherer G, Fischer G, Drakenberg T: Kinetic analysis of cyclophilin-catalyzed prolyl cis/trans isomerization by dynamic NMR spectroscopy. Biochemistry 1995, 34: 13594-13602
25 Schellenberger A: Enzymkatalyse: Einfuehrung in die Chemie, Biochemie und Technologie der Enzyme. Berlin [i.a.], Springer, 1989
26 Remington's Pharmaceutical Sciences, 1990, 18th ed., Mack Publishing Co., Easton, Pa.
27 Camps P, Perez F, Soldevilla N: (R)- and (S)-3-Hydroxy-4,4-dimethyl-1-phenyl-2-pyrolidinone as chiral auxilaries for the asymmetric synthesis of Alpha-hydroxy acids. Tetrahedron Asymmetry 1997, 8: 1877.
28 Villemin D, Hammadi M: A process using only water as solvent is described for the synthesis of aryloxyacetic acids under microwave irradiation. M, Syn Commun 1996, 26: 4337.
29 Hammond M L, Zambias R A, Chang M N: Antioxidant-based inhibitors of leukotriene biosynthesis. The discovery of 6- and 2 over black square]; [1 and 2 over black square]-[2-(hydroxymethyl)phenyl]-1-propen-3-yl]-2,3-dihydro-5-benzofuranol, a potent topical anti-inflammatory agent. J med chem. 1990, 33, 908.
30 Khosla M. C, Anand N: A rearrangement during the Cyclization of a-Phenoxy-phenylacetic Acid. Indian Journal of Chemistry, 1965, 3, 232
31 Mosmann T: Rapid calorimetric assay for cellular growth and survival: application to proliferation and cytotoxicity assays. J Immunol Meth 1983, 65:55-63.

1. A spiro derivative of the general formula (1) in all its enantiomeric forms: 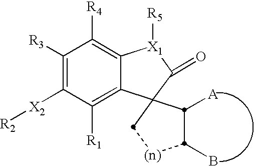
in which
X₁ represents —CH, —O— or nitrogen;
X₂ represents —CH₂, —O—, —NH—;
R₂ and R₅ represent a hydrogen atom or a linear or a branched C₁-C₈ alkyl group, which can be substituted by —OH, —OCH₃, —CH₂CH₂OCH₃, OCH₂CH₂—N(CH₃)₂ or by NH₂—, C₁-C₈ alkylamino or C₁-C₈ dialkylamino groups;
or the moiety X₁-R₅ represents —O;
R₁, R₃ and R₄ each independently represent a hydrogen atom, a linear or branched C₁-C₈ alkyl group, a linear or branched C₂-C₈ alkenyl group with one or several double bonds or a linear or branched C₂-C₈ alkinyl group with one or several triple bonds, a halogen atom selected from Cl, Br, I and F, a linear or branched C₁-C₈ acyl group or a C₁-C₈ amidoacyl group having the formula H₂NC(O)— or —HNC(O)—;
R₃ and R₄ and R₄ and R₅ can, additionally, together form a 5- or 6-membered aromatic or heteroaromatic ring, i.e. a ring substituted with nitrogen, oxygen or sulphur, which can additionally contain F, Cl, Br, I, CN, NO₂, —SH, O and —C(O)H;
(n) and the broken line represent a 4- (n=0), 5- (n=1), 6- (n=2) or 7- (n=3)-membered ring, wherein, except for the spiro carbon atom, each of the ring atoms . can individually and independently from each other represent a C-, N- or O-atom and these atoms can, except for oxygen, be linked by single as well as by double bonds, if C is the ring atom, it can be substituted by a linear or branched C₁-C₈ alkyl group, a linear or branched C₂-C₈ alkenyl group with one or several double bonds, a linear or branched C₂-C₈ alkinyl group with one or several triple bonds, a keto group, —OH, —OCH₃, —CH₂CH₂OCH₃, OCH₂CH₂—N(CH₃)₂ or by NH₂—, C₁-C₈ alkylamino or C₁-C₈ dialkylamino groups, if N is the ring atom, it can additionally have a hydrogen atom or it can be substituted by amino-, C₁-C₈ alkylamino or dialkylamino groups;
A and B represent either H or a 5- to 7-membered aromatic, heteroaromatic or saturated ring system; as well as pharmaceutically acceptable salts thereof.

2. A spiro derivative of the general formula (2) in all its enantiomeric forms: 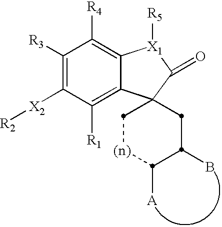
in which R₁, R₂, R₃, R₄, R₅, X₁, X₂, A and B are as defined in claim 1;
(n) and the broken line represent a 5- (n=0), 6- (n=1), 7- (n=2), or 8- (n=3)-membered ring, wherein, except for the spiro carbon atom, each of the ring atoms . can individually and independently from each other represent a C-, N- or O-atom and these atoms can, except for oxygen, be linked by single as well as by double bonds, if C is the ring atom, it can be substituted by a linear or branched C₁-C₈ alkyl group or a linear or branched C₂-C₈ alkenyl group with one or several double bonds, a linear or branched C₂-C₈ alkinyl group with one or several triple bonds, a keto group, —OH, —OCH₃, —CH₂CH₂OCH₃, OCH₂CH₂—N(CH₃)₂ or by NH₂—, C₁-C₈ alkylamino or C₁-C₈ dialkylamino groups, if N is the ring atom, it can additionally have a hydrogen atom or it can be substituted by amino-, C₁-C₈ alkylamino or dialkylamino groups as well as pharmaceutically acceptable salts thereof.

3. A carboxylic acid derivative of the general formula (3) in all its enantiomeric forms: 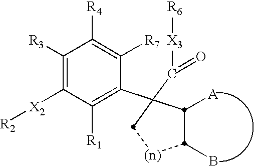
in which
X₃ represents oxygen or NH;
X₂ represents —CH₂, —O— or —NH—;
R₁, R₂, R₃, R₄, (n), A and B are as defined in claim 1;
R₆ and R₇ each independently represent a hydrogen atom, a linear or branched C₁-C₈ alkyl group, a linear or branched C₂-C₈ alkenyl group with one or several double bonds, or a linear or branched C₂-C₈ alkinyl group with one or several triple bonds, a halogen atom selected from Cl, Br, I and F, a linear or branched C₁-C₈ acyl group, or a C₁-C₈ amidoacyl group having the formula H₂NC(O)— or —HNC(O)—;
R₃ and R₄ and R₄ and R₇ can, additionally, together form a 5- or 6-membered aromatic or heteroaromatic ring, i.e. a ring substituted with nitrogen, oxygen or sulphur, which can additionally contain F, Cl, Br, I, CN, NO₂, —SH, O and —C(O)H as well as pharmaceutically acceptable salts thereof.

4. A carboxylic acid derivative of the general formula (4) and in all its enantiomeric forms: 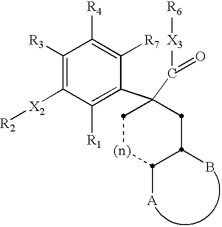
in which X₂, X₃, R₁, R₂, R₃, R₄, R₆, R₇, A and B are as defined in claim 3;
(n) is as defined in claim 2 as well as pharmaceutically acceptable salts thereof.

5. A Spiro derivative of the general formula (5) in all its enantiomeric forms: 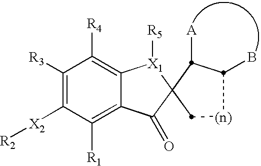
in which
X₁, X₂, R₁, R₂, R₃, R₄, R₅, (n), A and B are as defined in claim 1.

6. A Spiro derivative of the general formula (6) in all its enantiomeric forms: 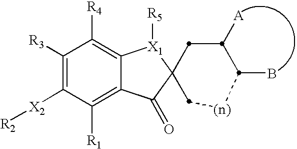
in which
X₁, X₂, R₁, R₂, R₃, R₄, R₅, A, B and (n) are as defined in claim 2 as well as pharmaceutically acceptable salts.

7. A ketone derivative of the general formula (7) in all its enantiomeric forms: 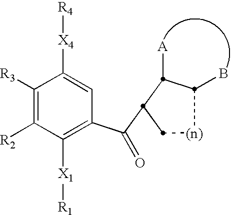
in which
X₁ and X₄ represent —CH₂, —O— or —NH—;
R₁ and R₄ represent a hydrogen atom or a linear or branched C₁-C₈ alkyl group, which can be substituted by —OH, —OCH₃, —CH₂CH₂OCH₃, OCH₂CH₂—N(CH₃)₂ or by NH₂—, C₁-C₈ alkylamino or C₁-C₈ dialkylamino groups;
R₂ represents independently a hydrogen atom, a linear or branched C₁-C₈ alkyl group, a linear or branched C₂-C₈ alkenyl group with one or several double bonds or a linear or branched C₂-C₈ alkinyl group with one or several triple bonds, a halogen atom selected from Cl, Br, I and F, a linear or branched C₁-C₈ acyl group, or a C₁-C₈ amidoacyl group having the formula H₂NC(O)— or —HNC(O)—;
R₂ and R₃ can, additionally, together form a 5- or 6-membered aromatic or heteroaromatic ring, i.e. a ring substituted with nitrogen, oxygen or sulphur, which can additionally contain F, Cl, Br, I, CN, NO₂, —SH, O and —C(O)H; and
R₃, (n), A and B are as defined in claim 1 as well as pharmaceutically acceptable salts thereof.

8. A ketone derivative of the general formula (8) in all its enantiomeric forms: 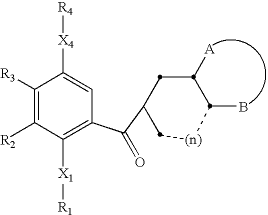
in which
X₁, X₄, R₁, R₂ and R₄ are as defined in claim 7;
R₃, (n), A and B are as defined in claim 2 as well as pharmaceutically acceptable salts thereof.

9. The compound according to claim 1, 2, 3, 4, 5, 6, 7 and 8 having a molecular weight lower than about 1,000 g/mol.

10. The compound according to claim 9 having an affinity to (PO₃H₂)Ser/(PO₃H₂)Thr-Pro-specific peptidyl-prolyl-cis/trans-isomerases of the parvuline family.

11. The compound according to claim 9, which can inhibit the peptidyl-prolyl-cis/trans-isomerase activity of (PO₃H₂)Ser/(PO₃H₂)Thr-Pro-specific peptidyl-prolyl-cis/trans-isomerases of the parvuline family.

12. Pharmaceutical composition comprising a compound according to any one of claims 1 to 11, which is optionally linked to a pharmacologically suitable carrier.

13. Use of a compound according to any one of claims 1 to 11, which is optionally linked to a pharmacologically acceptable carrier for the preparation of a pharmaceutical composition for inhibiting the isomerase activity of (PO₃H₂)Ser/(PO₃H₂)Thr-Pro-specific peptidyl-prolyl-cis/trans-isomerases of the parvuline family.

14. Use of a compound according to any one of claims 1 to 11, which is optionally linked to a pharmacologically suitable carrier for the preparation of a pharmaceutical composition for the prophylaxis or therapy of cell proliferative functional disorders in a mammal.

15. Use according to claim 14, wherein the cell proliferative funcational disorders are selected from
inflammatory autoimmune diseases, bacterial infections, viral infections, diseases caused by parasites and protozoa, cancer, fibrosing diseases, non-neoplastic changes and diseases which are caused by prions and changes in the structure of cellular proteins.

16. Use according to claim 15, wherein the inflammatory autoimmune disease is selected from psoriasis, neurodermatitis, systemic lupus erythematosus, glomerulonephritis, multiple sclerosis, Basedow's disease, chronic thyreoiditis, myasthenia gravis, pemphigus, dermatosclerosis, colitis ulcerosa, rheumatoid arthritis, ITP, haemolytic anaemia, diabetes mellitus type 1, uveitis, Cogan's syndrome.

17. Use according to claim 15, wherein the bacterial infections are caused by Bacillus, Camphylobacter, Clostridium, Diplococcus, Enterobacter, Enterococcus, Erysipelothricosis, Escherichia, Hemophilus, Klebsiella, Listeria, Morganella, Mycobacterium, Neisseria, Proteus, Providencia, Salmonella, Serratia, Shigella, Staphylococcus, Streptococcus or Yersinia.

18. Use according to claim 15, wherein the viral infections are caused by adeno viruses, arboviruses, bunya viruses, dengue viruses, flavi viruses, herpes viruses paramyxo viruses, picoma viruses, polyoma viruses, orbiviruses, orthomyxo viruses, rhabdo viruses, retro viruses, rubella viruses or toga viruses.

19. Use according to claim 15, wherein the infections caused by fungi affect the whole organism, the skin or the urogenital tract and are caused by Absidia, Aspergillus, Candida, Coccidioides, Cryptococcus, Blastocyces, Histoplasma, Hormodendrum, Mucor, Nocardia, Paracoccidioides, Phialopora, Rhinosporidium Rhizopus, Sporothrix, Microsporum, Trichophyton, Epidermophyton, Candida, Zygomycodoides and Pityrosporum.

20. Use according to claim 15, wherein the diseases caused by parasites and protozoa are selected from brucellosis, cholera, diarrhea, gastroenteritis, gonorrhea, Lyme disease, mastoiditis, meningitis, anthrax, pneumoniae, rheumatic fever, dysentery, tetanus, tuberculosis and typhus.

21. Use according to claim 15, wherein the cancer is selected from hematopoetic changes as leukaemias and lymphomas; carcinomas, sarcomas, osteomas, fibrosarcomas and chondrosarcomas.

22. Use according to claim 15, wherein the fibrosing diseases are selected from fibromyalgia, fibroses, fibromuscular hyperplasia, restenosis and arteriosclerosis.

23. Use according to claim 15, wherein the non-neoplastic changes are selected from prostate hypertrophy, endometriosis and psoriasis.

24. Use according to claim 15, wherein the diseases which are caused by prions and changes in the structure of cellular proteins are selected from Alzheimer's disease, Creutzfeldt-Jacob disease, and its new variant, the nvCreutzfeldt-Jacob disease, scrapie, kuru, fatal familial insomnia and the Gerstmann-Straussler syndrome.

25. Use according to any one of claims 13 to 24, wherein the mammal is human.
